SEQUENCE LISTING (78 sequences)

SEQ ID NO: 1 - DNA, 32 bp, Artificial Sequence
  Synthesized ADH1 upstream forward primer
cgggatccactgtagccctagacttgatagcc

SEQ ID NO: 2 - DNA, 44 bp, Artificial Sequence
  Synthesized ADH1 upstream reverse primer
ataagaatgcggccgctgtatatgagatagttgattgtatgctt

SEQ ID NO: 3 - DNA, 37 bp, Artificial Sequence
  Synthesized ADH1 downstream forward primer
gactagtgcgaatttcttatgatttatgatttttatt

SEQ ID NO: 4 - DNA, 37 bp, Artificial Sequence
  Synthesized ADH1 downstream reverse primer
acatgccatggaagcatgcacgtatacacttgagtaa

SEQ ID NO: 5 - DNA, 35 bp, Artificial Sequence
  Synthesized PDC1 upstream forward primer
cgggatccattatgtatgctcttctgacttttcgt

SEQ ID NO: 6 - DNA, 44 bp, Artificial Sequence
  Synthesized PDC1 upstream reverse primer
ataagaatgcggccgctttgattgatttgactgtgttattttgc

SEQ ID NO: 7 - DNA, 37 bp, Artificial Sequence
  Synthesized PDC1 downstream forward primer
cgggatccgcgatttaatctctaattattagttaaag

SEQ ID NO: 8 - DNA, 44 bp, Artificial Sequence
  Synthesized PDC1 downstream reverse primer
ataagaatgcggccgctttcaatcattggagcaatcattttaca

SEQ ID NO: 9 - DNA, 50 bp, Artificial Sequence
  Synthesized DLD1-HIS3 upstream linking primer
gcgtagttggccccaactggtgcagtaatacgttttaagagcttggtgag

SEQ ID NO: 10 - DNA, 50 bp, Artificial Sequence
  Synthesized DLD1-HIS3 downstream linking primer
cgtgaagggtgaaaaaggaaaatcagatacctacataagaacacctttgg

SEQ ID NO: 11 - DNA, 22 bp, Artificial Sequence
  Synthesized F_PDC5_UP_676 primer
gtcagcattgacacgttcgatt

SEQ ID NO: 12 - DNA, 39 bp, Artificial Sequence
  Synthesized R_KlURA3-PDC5_UP primer
tctacccagaatcacttctttcgagagattgtcataatc

SEQ ID NO: 13 - DNA, 38 bp, Artificial Sequence
  Synthesized F_PDC5_UP-AL_KlURA3 primer
caatctctcgaaagaagtgattctgggtagaagatcgg

SEQ ID NO: 14 - DNA, 27 bp, Artificial Sequence
  Synthesized R_AL_KlURA3 primer
gagcaatgaacccaataacgaaatctt

SEQ ID NO: 15 - DNA, 24 bp, Artificial Sequence
  Synthesized F_BR_KlURA3 primer
cttgacgttcgttcgactgatgag

SEQ ID NO: 16 - DNA, 22 bp, Artificial Sequence
  Synthesized R_PDC5_DOWN_522 primer
caagtcaaccaagttagctggc

SEQ ID NO: 17 - DNA, 43 bp, Artificial Sequence
  Synthesized R_SCO1p-BR_KlURA3 primer
ctctcctaatagacgtggtgtcaccatgaacgacaattcttaa

SEQ ID NO: 18 - DNA, 38 bp, Artificial Sequence
  Synthesized F_SCO1p_500 primer
cgttcatggtgacaccacgtctattaggagagccattc

SEQ ID NO: 19 - DNA, 41 bp, Artificial Sequence
  Synthesized R_PDC5_DOWN_500-SCO1p primer
aaggttatttcagacatcttttctacgtttgctgttttttc

SEQ ID NO: 20 - DNA, 42 bp, Artificial Sequence
  Synthesized F_SCO1p-PDC5_DOWN_500 primer
cagcaaacgtagaaaagatgtctgaaataaccttaggtaaat

SEQ ID NO: 21 - DNA, 44 bp, Artificial Sequence
  Synthesized R_SCO2p-BR_KlURA3 primer
atcgaataagtaacaagcgtgtcaccatgaacgacaattcttaa

SEQ ID NO: 22 - DNA, 38 bp, Artificial Sequence
  Synthesized F_SCO2p_500 primer
cgttcatggtgacacgcttgttacttattcgataacgc

SEQ ID NO: 23 - DNA, 40 bp, Artificial Sequence
  Synthesized R_PDC5_DOWN_500-SCO2p primer
aaggttatttcagacattttactctcgcttcccaaattcc

SEQ ID NO: 24 - DNA, 40 bp, Artificial Sequence
  Synthesized F_SCO2p-PDC5_DOWN_500 primer
ggaagcgagagtaaaatgtctgaaataaccttaggtaaat

SEQ ID NO: 25 - DNA, 47 bp, Artificial Sequence
  Synthesized R_IDP2p-BR_KlURA3 primer
taaaaataaatagatagacgtgtgtcaccatgaacgacaattcttaa

SEQ ID NO: 26 - DNA, 42 bp, Artificial Sequence
  Synthesized F_IDP2p_500 primer
cgttcatggtgacacacgtctatctatttatttttataactc

SEQ ID NO: 27 - DNA, 44 bp, Artificial Sequence
  Synthesized R_PDC5_DOWN_500-IDP2p primer
aaggttatttcagacattacgattttatatatatacgtacgtta

SEQ ID NO: 28 - DNA, 45 bp, Artificial Sequence
  Synthesized F_IDP2p-PDC5_DOWN_500 primer
cgtatatatataaaatcgtaatgtctgaaataaccttaggtaaat

SEQ ID NO: 29 - DNA, 43 bp, Artificial Sequence
  Synthesized R_ACS1p-BR_KlURA3 primer
ctggacgtatgtgcacagtgtcaccatgaacgacaattcttaa

SEQ ID NO: 30 - DNA, 38 bp, Artificial Sequence
  Synthesized F_ACS1p_500 primer
cgttcatggtgacactgtgcacatacgtccagaatgat

SEQ ID NO: 31 - DNA, 39 bp, Artificial Sequence
  Synthesized R_PDC5_DOWN_500-ACS1p primer
aaggttatttcagacatagcacagtgggcaatgtctttc

SEQ ID NO: 32 - DNA, 41 bp, Artificial Sequence
  Synthesized F_ACS1p-PDC5_DOWN_500 primer
cattgcccactgtgctatgtctgaaataaccttaggtaaat

SEQ ID NO: 33 - DNA, 45 bp, Artificial Sequence
  Synthesized R_FBA1p-BR_KlURA3 primer
ttatttacgtaatgacccagtgtcaccatgaacgacaattcttaa

SEQ ID NO: 34 - DNA, 40 bp, Artificial Sequence
  Synthesized F_FBA1p_500 primer
cgttcatggtgacactgggtcattacgtaaataatgatag

SEQ ID NO: 35 - DNA, 44 bp, Artificial Sequence
  Synthesized R_PDC5_DOWN_500-FBA1p primer
aaggttatttcagacattttgaatatgtattacttggttatggt

SEQ ID NO: 36 - DNA, 45 bp, Artificial Sequence
  Synthesized F_FBA1p-PDC5_DOWN_500 primer
ccaagtaatacatattcaaaatgtctgaaataaccttaggtaaat

SEQ ID NO: 37 - DNA, 41 bp, Artificial Sequence
  Synthesized F-ALPDC5-BamHI primer
gagctcggatccaaggaaataaagcaaataacaataacacc

SEQ ID NO: 38 - DNA, 43 bp, Artificial Sequence
  Synthesized R-ALPDC5-NotI primer
accatggcggccgctttgttcttcttgttattgtattgtgttg

SEQ ID NO: 39 - DNA, 42 bp, Artificial Sequence
  Synthesized F-BRPDC5-SpeI primer
ggatccactagtgctaattaacataaaactcatgattcaacg

SEQ ID NO: 40 - DNA, 42 bp, Artificial Sequence
  Synthesized R-BRPDC5-NcoI primer
cagctgccatggtattctaaataagatgtaaggccttgtaat

SEQ ID NO: 41 - DNA, 38 bp, Artificial Sequence
  Synthesized F_PDC1 primer
ataactagtatgtctgaaattactttgggtaaatattt

SEQ ID NO: 42 - DNA, 44 bp, Artificial Sequence
  Synthesized R_PDC1 primer
caaaggaaaaggggcctgtttattgcttagcgttggtagcagca

SEQ ID NO: 43 - DNA, 43 bp, Artificial Sequence
  Synthesized F_CYC1t primer
taccaacgctaagcaataaacaggccccttttcctttgtcgat

SEQ ID NO: 44 - DNA, 33 bp, Artificial Sequence
  Synthesized R_CYC1t primer
atactcgaggcaaattaaagccttcgagcgtcc

SEQ ID NO: 45 - DNA, 33 bp, Artificial Sequence
  Synthesized F_PDC1p primer
aaagagctccatgcgactgggtgagcatatgtt

SEQ ID NO: 46 - DNA, 37 bp, Artificial Sequence
  Synthesized R_PDC1p primer
ataactagttttgattgatttgactgtgttattttgc

SEQ ID NO: 47 - DNA, 37 bp, Artificial Sequence
  Synthesized F_IDP2p primer
aaagagctcacgtctatctatttatttttataactcc

SEQ ID NO: 48 - DNA, 37 bp, Artificial Sequence
  Synthesized R_IDP2p primer
ataactagttacgattttatatatatacgtacgttac

SEQ ID NO: 49 - DNA, 35 bp, Artificial Sequence
  Synthesized F_BamHI-PDC5_UP primer
cgggatccaggccaaggaaataaagcaaataacaa

SEQ ID NO: 50 - DNA, 44 bp, Artificial Sequence
  Synthesized R_NotI-PDC5_UP primer
ataagaatgcggccgctttgttcttcttgttattgtattgtgtt

SEQ ID NO: 51 - DNA, 36 bp, Artificial Sequence
  Synthesized F_BamHI-PDC5_DOWN primer
cgggatccgctaattaacataaaactcatgattcaa

SEQ ID NO: 52 - DNA, 44 bp, Artificial Sequence
  Synthesized R_NotI-PDC5_DOWN primer
ataagaatgcggccgctattctaaataagatgtaaggccttgta

SEQ ID NO: 53 - DNA, 27 bp, Artificial Sequence
  Synthesized F_PDC5_UP primer
aggccaaggaaataaagcaaataacaa

SEQ ID NO: 54 - DNA, 27 bp, Artificial Sequence
  Synthesized R_AL_KlURA3 primer
gagcaatgaacccaataacgaaatctt

SEQ ID NO: 55 - DNA, 24 bp, Artificial Sequence
  Synthesized F_BR_KlURA3 primer
cttgacgttcgttcgactgatgag

SEQ ID NO: 56 - DNA, 28 bp, Artificial Sequence
  Synthesized R_PDC5_DOWN primer
tattctaaataagatgtaaggccttgta

SEQ ID NO: 57 - DNA, 35 bp, Artificial Sequence
  Synthesized F_BamHI-PDC6_UP primer
cgggatcctgttatagagttcacaccttattcaca

SEQ ID NO: 58 - DNA, 45 bp, Artificial Sequence
  Synthesized R_NotI-PDC6_UP primer
ataagaatgcggccgctttgttggcaatatgtttttgctatatta

SEQ ID NO: 59 - DNA, 34 bp, Artificial Sequence
  Synthesized F_BamHI-PDC6_DOWN primer
cgggatccgccattagtagtgtactcaaacgaat

SEQ ID NO: 60 - DNA, 43 bp, Artificial Sequence
  Synthesized R_NotI-PDC6_DOWN primer
ataagaatgcggccgcgatgcagaatgagcacttgttatttat

SEQ ID NO: 61 - DNA, 27 bp, Artificial Sequence
  Synthesized F_PDC6_UP primer
tgttatagagttcacaccttattcaca

SEQ ID NO: 62 - DNA, 27 bp, Artificial Sequence
  Synthesized R_AL_KlURA3 primer
gagcaatgaacccaataacgaaatctt

SEQ ID NO: 63 - DNA, 24 bp, Artificial Sequence
  Synthesized F_BR_KlURA3 primer
cttgacgttcgttcgactgatgag

SEQ ID NO: 64 - DNA, 27 bp, Artificial Sequence
  Synthesized R_PDC6_DOWN primer
gatgcagaatgagcacttgttatttat

SEQ ID NO: 65 - DNA, 50 bp, Artificial Sequence
  Synthesized F_SpeI_ALD2 primer
caagctggccgctctagaactagtatgcctaccttgtatactgatatcga

SEQ ID NO: 66 - DNA, 50 bp, Artificial Sequence
  Synthesized R_XhoI_ALD2 primer
acataactaattacatgactcgagttagttgtccaaagagagatttatgt

SEQ ID NO: 67 - DNA, 50 bp, Artificial Sequence; identical to SEQ ID NO: 65
  Synthesized F_SpeI_ALD3 primer

SEQ ID NO: 68 - DNA, 50 bp, Artificial Sequence
  Synthesized R_XhoI_ALD3 primer
acataactaattacatgactcgagttatttatccaatgaaagatccacat

SEQ ID NO: 69 - DNA, 46 bp, Artificial Sequence
  Synthesized F_SpeI_ACS1 primer
tccaagctggccgctctagaactagtatgtcgccctctgccgtaca

SEQ ID NO: 70 - DNA, 50 bp, Artificial Sequence
  Synthesized R_EcoRI_ACS1 primer
tatcgataagcttgatatcgaattcttacaacttgaccgaatcaattaga

SEQ ID NO: 71 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 72 - protein, 563 aa, Saccharomyces cerevisiae
MSEITLGKYLFERLSQVNCNTVFGLPGDFNLSLLDKLYEVKGMRWAGNANELNAAYAADGYARIKGMSCIITTFGVGELSALNGIAGSYAEHVGVLHVVGVPSISSQAKQLLLHHTLGNGDFTVFHRMSANISETTAMITDIANAPAEIDRCIRTTYTTQRPVYLGLPANLVDLNVPAKLLETPIDLSLKPNDAEAEAEVVRTVVELIKDAKNPVILADACASRHDVKAETKKLMDLTQFPVYVTPMGKGAIDEQHPRYGGVYVGTLSRPEVKKAVESADLILSIGALLSDFNTGSFSYSYKTKNIVEFHSDHIKIRNATFPGVQMKFALQKLLDAIPEVVKDYKPVAVPARVPITKSTPANTPMKQEWMWNHLGNFLREGDIVIAETGTSAFGINQTTFPTDVYAIVQVLWGSIGFTVGALLGATMAAEELDPKKRVILFIGDGSLQLTVQEISTMIRWGLKPYIFVLNNNGYTIEKLIHGPHAEYNEIQGWDHLALLPTFGARNYETHRVATTGEWEKLTQDKDFQDNSKIRMIEVMLPVFDAPQNLVKQAQLTAATNAKQ

SEQ ID NO: 73 - protein, 563 aa, Saccharomyces cerevisiae
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVIRLIELKLPVFDAPESLIKQAQLTAATNAKQ

SEQ ID NO: 74 - protein, 506 aa, Saccharomyces cerevisiae
MPTLYTDIEIPQLKISLKQPLGLFINNEFCPSSDGKTIETVNPATGEPITSFQAANEKDVDKAVKAARAAFDNVWSKTSSEQRGIYLSNLLKLIEEEQDTLAALETLDAGKPYHSNAKGDLAQILQLTRYFAGSADKFDKGATIPLTFNKFAYTLKVPFGVVAQIVPWNYPLAMACWKLQGALAAGNTVIIKPAENTSLSLLYFATLIKKAGFPPGVVNIVPGYGSLVGQALASHMDIDKISFTGSTKVGGFVLEASGQSNLKDVTLECGGKSPALVFEDADLDKAIDWIAAGIFYNSGQNCTANSRVYVQSSIYDKFVEKFKETAKKEWDVAGKFDPFDEKCIVGPVISSTQYDRIKSYIERGKREEKLDMFQTSEFPIGGAKGYFIPPTIFTDVPQTSKLLQDEIFGPVVVVSKFTNYDDALKLANDTCYGLASAVFTKDVKKAHMFARDIKAGTVWINSSNDEDVTVPFGGFKMSGIGRELGQSGVDTYLQTKAVHINLSLDN

SEQ ID NO: 75 - protein, 506 aa, Saccharomyces cerevisiae
MPTLYTDIEIPQLKISLKQPLGLFINNEFCPSSDGKTIETVNPATGEPITSFQAANEKDVDKAVKAARAAFDNVWSKTSSEQRGIYLSNLLKLIEEEQDTLAALETLDAGKPFHSNAKQDLAQIIELTRYYAGAVDKFNMGETIPLTFNKFAYTLKVPFGVVAQIVPWNYPLAMACRKMQGALAAGNTVIIKPAENTSLSLLYFATLIKKAGFPPGVVNVIPGYGSVVGKALGTHMDIDKISFTGSTKVGGSVLEASGQSNLKDITLECGGKSPALVFEDADLDKAIEWVANGIFFNSGQICTANSRVYVQSSIYDKFVEKFKETAKKEWDVAGKFDPFDEKCIVGPVISSTQYDRIKSYIERGKKEEKLDMFQTSEFPIGGAKGYFIPPTIFTDVPETSKLLRDEIFGPVVVVSKFTNYDDALKLANDTCYGLASAVFTKDVKKAHMFARDIKAGTVWINQTNQEEAKVPFGGFKMSGIGRESGDTGVDNYLQIKSVHVDLSLDK

SEQ ID NO: 76 - protein, 713 aa, Saccharomyces cerevisiae
MSPSAVQSSKLEEQSSEIDKLKAKMSQSAATAQQKKEHEYEHLTSVKIVPQRPISDRLQPAIATHYSPHLDGLQDYQRLHKESIEDPAKFFGSKATQFLNWSKPFDKVFIPDPKTGRPSFQNNAWFLNGQLNACYNCVDRHALKTPNKKAIIFEGDEPGQGYSITYKELLEEVCQVAQVLTYSMGVRKGDTVAVYMPMVPEAIITLLAISRIGAIHSVVFAGFSSNSLRDRINDGDSKVVITTDESNRGGKVIETKRIVDDALRETPGVRHVLVYRKTNNPSVAFHAPRDLDWATEKKKYKTYYPCTPVDSEDPLFLLYTSGSTGAPKGVQHSTAGYLLGALLTMRYTFDTHQEDVFFTAGDIGWITGHTYVVYGPLLYGCATLVFEGTPAYPNYSRYWDIIDEHKVTQFYVAPTALRLLKRAGDSYIENHSLKSLRCLGSVGEPIAAEVWEWYSEKIGKNEIPIVDTYWQTESGSHLVTPLAGGVTPMKPGSASFPFFGIDAVVLDPNTGEELNTSHAEGVLAVKAAWPSFARTIWKNHDRYLDTYLNPYPGYYFTGDGAAKDKDGYIWILGRVDDVVNVSGHRLSTAEIEAAIIEDPIVAECAVVGFNDDLTGQAVAAFVVLKNKSSWSTATDDELQDIKKHLVFTVRKDIGPFAAPKLIILVDDLPKTRSGKIMRRILRKILAGESDQLGDVSTLSNPGIVRHLIDSVKL

SEQ ID NO: 77 - protein, 348 aa, Saccharomyces cerevisiae
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 78 - protein, 587 aa, Saccharomyces cerevisiae
MLWKRTCTRLIKPIAQPRGRLVRRSCYRYASTGTGSTDSSSQWLKYSVIASSATLFGYLFAKNLYSRETKEDLIEKLEMVKKIDPVNSTLKLSSLDSPDYLHDPVKIDKVVEDLKQVLGNKPENYSDAKSDLDAHSDTYFNTHHPSPEQRPRIILFPHTTEEVSKILKICHDNNMPVVPFSGGTSLEGHFLPTRIGDTITVDLSKFMNNVVKFDKLDLDITVQAGLPWEDLNDYLSDHGLMFGCDPGPGAQIGGCIANSCSGTNAYRYGTMKENIINMTIVLPDGTIVKTKKRPRKSSAGYNLNGLFVGSEGTLGIVTEATVKCHVKPKAETVAVVSFDTIKDAAACASNLTQSGIHLNAMELLDENMMKLINASESTDRCDWVEKPTMFFKIGGRSPNIVNALVDEVKAVAQLNHCNSFQFAKDDDEKLELWEARKVALWSVLDADKSKDKSAKIWTTDVAVPVSQFDKVIHETKKDMQASKLINAIVGHAGDGNFHAFIVYRTPEEHETCSQLVDRMVKRALNAEGTCTGEHGVGIGKREYLLEELGEAPVDLMRKIKLAIDPKRIMNPDKIFKTDPNEPANDYR

(19) United States
(12) Patent Application Publication
(10) Pub. No.: US 2018/0201960 A1
(43) Pub. Date: Jul. 19, 2018

(54) MICROORGANISM HAVING ENHANCED PRODUCTIVITY OF LACTIC ACID AND A PROCESS FOR PRODUCING LACTIC ACID USING THE SAME

(71) Applicant: CJ CHEILJEDANG CORPORATION, Seoul (KR)

(72) Inventors: Eun Bin Yang, Seoul (KR); Tae Hee Lee, Gyeonggi-do (KR); Seon Hye Kim, Gyeonggi-do (KR); Gyu Hyeon Song, Seoul (KR); Cheol Woong Ha, Seoul (KR); Kyung Su Na, Gyeonggi-do (KR); Young Lyeol Yang, Seoul (KR); Min Sun Kang, Seoul (KR); Hyo Hyoung Lee, Incheon (KR)

(21) Appl. No.: 15/918,995

(22) Filed: Mar. 12, 2018

Related U.S. Application Data
(63) Continuation of application No. 14/783,012, filed on Oct. 7, 2015, now abandoned.

(30) Foreign Application Priority Data
May 9, 2014 (KR) 10 -2014-0055865

(51) Int. Cl. C12P 7/56; C12N 9/04; C12N 9/88; C12N 9/02; C12N 9/00

(52) U.S. Cl. CPC C12P 7/56; C12Y 602/01001; C12Y 401/01001; C12N 9/93; C12N 9/0006; C12N 9/88; C12N 9/0008; C12Y 101/01028

(57) ABSTRACT

The present invention relates to Saccharomyces sp. capable of producing lactic acid with a decreased activity of pyruvate decarboxylase (PDC) and increased activities of aldehyde dehydrogenase (ALD) and acetyl-CoA synthetase (ACS), and a method of producing lactic acid from the culture medium obtained by culturing the microorganism.

### CROSS-REFERENCE TO RELATED APPLICATIONS

This application is a continuation of U.S. application Ser. No. 14/783,012, filed May 8, 2015, which is a U.S. national phase application of International PCT Patent Application No. PCT/KR2015/004600, which was filed on May 8, 2015, which claims priority to Korean Patent Application Nos. 10-2014-0055865, filed May 9, 2014. These applications are incorporated herein by reference in their entireties.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is HANO_036_01US_SeqList_ST25.txt. The text file is 48 KB, was created on Mar. 9, 2018, and is being submitted electronically via EFS-Web.

### TECHNICAL FIELD

The present invention relates to a lactic acid-producing recombinant Saccharomyces sp. microorganism, and a method for producing lactic acid from the culture medium containing the microorganism by culturing the same.

### BACKGROUND ART

Generally, lactic acid is an important organic acid with a wide range of applications including food additives such as food preservative, fragrance, or acidifier etc., and has been used broadly for industrial purposes such as cosmetics, chemistry, metals, electronics, fabrics, dyeing textiles, and pharmaceutical industries, etc. In addition, lactic acid is an essential ingredient of polylactic acid, one of biodegradable plastics, and thus the demand for lactic acid has been increasing significantly. It is also used as an important material for the productions of many chemical compounds including polylactic acid, acetaldehyde, polypropylene glycol, acrylic acid, 2,3-pentathione, etc. Specifically, D-type lactic acid is an essential ingredient for producing streocomplex PLA, which is an optical isomer required for the production of highly heat-resistant PLA.

Specifically, the method for producing lactic acid includes a traditional chemical synthesis and a biological fermentation process. When lactic acid is produced via the chemical synthesis, lactic acid is produced in the form of a racemic mixture consisting of 50% D-type lactic acid and 50% L-type lactic acid, and it is difficult to control the composition ratio, and thus polylactic acid produced therefrom may become an amorphous polymer having a low melting point, thereby imposing limitations on the development of their use. On the other hand, the biological fermentation process allows to selectively produce D-type lactic acid or L-type lactic acid depending on the strain used. Thus, the latter is preferred commercially because it is possible to produce a particular isoform of lactic acid.

Meanwhile, attempts have been made in order to improve the productivity of lactic acid via various gene manipulations using a saccharomyces sp. microorganism having D-lactic acid—producing ability, by introducing a gene of an enzyme for conversion into D-type lactic acid. Specifically, attempts have been made to improve the productivity of lactic acid by strengthening the activity of lactic acid dehydrogenase (LDH) while decreasing the activities of pyruvate decarboxylase (PDC), aldehyde dehydrogenase (ALD), and/or acetyl-CoA synthetase (ACS), and (U.S. Patent Application Publication Nos. 2012-021421, 2010-0248233, and 2006-0148050). However, the overall fermentation productivity was low due to the low cell growth of the lactic acid-producing strain.

Accordingly, intensive efforts have been made by the present inventors in order to obtain a microorganism having improved lactic acid productivity with an efficient cell growth while decreasing the activity of PDC. As a result, it has been confirmed that strains, in which the activities of PDC isotypes were controlled and the activities of aldehyde dehydrogenase and acetyl-CoA were increased, were able to increase the lactic acid production yield and facilitate the cell growth of the strains, thereby improving the overall lactic acid fermentation productivity, and this has led to the completion of the present invention.

### DISCLOSURE

### Technical Problem

An objective of the present invention is to provide a Saccharomyces sp. microorganism having improved productivity of lactic acid.

Another objective of the present invention is to provide a method for producing lactic acid using the Saccharomyces sp. microorganism.

### Technical Solution

In a first aspect of the present invention, to achieve the objectives described above, there is provided a Saccharomyces sp. microorganism having improved productivity of lactic acid, in which the microorganism is mutated so that (a) the activity of pyruvate decarboxylase is decreased compared to that of a non-mutated lactic acid-producing strain; and (b) the activities of aldehyde dehydrogenase and acetyl-CoA synthetase are improved compared to that of a non-mutated lactic acid-producing strain.

Generally, a lactic acid-producing Saccharomyces sp. microorganism produces lactic acid via lactic acid dehydrogenase (LDH) using pyruvate as a substrate. Ethanol fermentation pathway and acetyl-CoA production pathway, the representative metabolic pathways utilizing pyruvate as a common substrate, were blocked. Decreasing PDC activity may be helpful in the production of lactic acid and the yield improvement thereof, however, when the level of decrease reached a certain level, insufficient amount of cytosolic acetyl-CoA was produced, which in turn, blocked the cell growth, and thus the normal fermentation was not achieved. Accordingly, the present inventor developed a Saccharomyces sp. microorganism having improved productivity of lactic acid by improving the overall lactic acid fermentation productivity, in which the growth rate of the microorganism was maintained with improved lactic acid productivity yield by regulating the acetyl-CoA pathway at a minimum level.

The term “pyruvate decarboxylase (PDC) used herein refers to a protein having an activity capable of mediating a reaction responsible for producing carbonic acid and acetaldehyde from pyruvate, but is not limited to any derivative thereof or an isotype having the same activity. The protein has been known to be involved in a step of alcohol fermentation, and is mostly present in yeasts and plants. The pyruvate decarboxylase of the present invention may be intrinsically present in a Saccharomyces sp. microorganism, or may be PDC1, PDC5, and/or PDC6, or specifically PDC1, PDC5, and/or PDC6 of Saccharomyces cerevisiae, but is not limited thereto. The protein may include any variants or analogues thereof as long as they are biologically identical and have corresponding activities to the protein. The amino acid sequences of the protein may be obtained from a known database, etc., e.g., GenBank of NCBI, etc., but is not limited thereto. Specifically, PDC1 may consist of an amino acid sequence of SEQ ID NO: 71, PDC5 of an amino acid sequence of SEQ ID NO: 72, and PDC6 of an amino acid sequence of SEQ ID NO: 73. The protein may include amino acid sequences having a homology of more than 70%, specifically more than 80%, more specifically more than 90%, and even more specifically more than 95%, to each of the above-listed amino acid sequences. Any variant of the above-listed sequences encoding the same amino acid sequences, which results from genetic code degeneracy, may also be included in the present invention.

The term “homology” used herein refers to a degree of similarity between a plurality of nucleotide sequences or amino acid sequences, and is a unit representing a sequence having the same sequences to the amino acid sequences or the nucleotide sequences of the present invention, with a probability equal to or greater than the above probability. Such homology may be determined by comparing the two given sequences with the naked eye, but rather, it may be measured using a sequence comparison program, which is easily accessible, that interprets the degree of homology by arranging the sequences to be compared side by side. The sequence comparison programs known in the art include FASTP, BLAST, BLAST2, PSIBLAST, and a software containing CLUSTAL W, etc.

Numerous examples have been reported regarding the production of lactic acid by allowing a defect in PDC1, whose exhibits a major activity in lactic acid production (Appl Microbiol Biotechnol. 2009, 82(5):883-90). In this case, since PDC6 is rarely expressed, the actual PDC activity as appears to be due to the expression of PDC5 gene. According to the reports, the defectin PDC1 alone does not hinder the cell growth of a wild-type strain, and also about 60-70% of PDC activity can be maintained compared to that of the wild-type, and thus no significant phenotypic change has been observed in the strain (J Bacteriol. 1990, 172(2):678-685).

As an alternative, a strain having a simultaneous double defect in both PDC1 and PDC5 genes, which exhibit major PDC activities in yeasts, may be prepared. In such case, lactic acid fermentation can be carried out using a sugar source such as glycogen in the absence of a co-substrate such as acetic acid or ethanol. However, it resulted in decrease in the growth rate of the yeast strain due to a rapid decrease in PDC activity, thereby reducing the fermentation productivity of lactic acid (Biosci Biotechnol Biochem. 2006, 70(5): 1148-1153).

Meanwhile, in order to maximize the lactic acid via LDH pathway, which competes with PDC for pyruvate, a strain with a simultaneous triple defect in PDC1, PDC5, and PDC6 may be prepared. In this case, lactic acid fermentation yield may be maximized but the metabolic capabilities of ethanol and acetic acid due to catabolite repression induced in the presence of glucose may be further inhibited, thereby reducing cell growth and ultimately leading to decrease in fermentation productivity (Curr Genet. 2003, 43(3): 139-160).

Specifically, the decrease in pyruvate decarboxylase (PDC) activity of the present invention may i) inactivate PDC1 activity and decrease PDC5 activity; or ii) decrease PDC1 activity and inactivate PDC5 activity.

In an exemplary embodiment of the present invention, four different strains, which include a strain with a decreased PDC5 activity by substituting the promoter of PDC5 gene, a strain that caused a defect in PDC5 gene t by recovering PDC1 activity, a strain that caused a double defect in PDC1 and PDC5 genes, and a strain that caused a triple defect in PDC1, PDC5, and PDC6 genes, were prepared based on a Saccharomyces cerevisiae strain in which PDC1 activity was inactivated. Among the thus prepared strains, the strain having a triple gene defect was shown to rarely undergo cell growth.

The term “aldehyde dehydrogenase (ALD)” used herein refers to a protein having an activity of mainly producing acetic acid from acetaldehyde as a protein having an activity of producing carboxylic acid or an acyl group by the oxidation of aldehyde, but is not limited to a derivative thereof or an isotype having the same activity, in the present invention. The aldehyde dehydrogenase of the present invention may be derived from a Saccharomyces sp. microorganism, or may be ALD2 and/or ALD3. Specifically, the protein may be ALD2 and/or ALD3 of Saccharomyces cerevisiae, but is not limited thereto, and may include any variant or an analogue thereof as long as they are biologically identical and have corresponding activities to the protein. The amino acid sequences of the protein may be obtained from database, etc., known in the art, e.g., GenBank of NCBI, etc., but is not limited thereto. Specifically, ALD2 may consist of an amino acid sequence of SEQ ID NO: 74, and ALD3 may consist of an amino acid sequence of SEQ ID NO: 75. The protein may include amino acid sequences having a homology of more than 70%, specifically more than 80%, more specifically more than 90%, and even more specifically more than 95%, to the amino acid sequences. Any variant of the sequences encoding the identical amino acid sequences, which results from genetic code degeneracy, may also be included in the present invention.

The term “acetyl-CoA synthetase (ACS) used herein refers to a protein having an activity of catalyzing the thioesterification of acetic acid and CoA in conjugation with an ATP decomposition reaction, but is not limited to a derivative or an isotype having the same activity in the present invention. It has been known that the protein is present in microorganisms, plants, and animals, etc. The acetyl-CoA synthetase of the present invention may be derived from a Saccharomyces sp. microorganism or may be ACS1. Specifically, the protein may be ACS1 of Saccharomyces cerevisiae, but is not limited thereto, and may include any variant or an analogue thereof as long as they are biologically identical and have corresponding activities to the protein. The amino acid sequences of the protein may be obtained from a known database, etc., e.g., GenBank of NCBI, etc., but is not limited thereto. Specifically, ACS1 may be composed of an amino acid sequence of SEQ ID NO: 76, and may include amino acid sequences having a homology of more than 70%, specifically more than 80%, more specifically more than 90%, and even more specifically more than 95%, to the amino acid sequence. A protein mutant of the sequence encoding the identical amino acid sequences, which results from genetic code degeneracy, may also be included in the present invention.

In an exemplary embodiment of the present invention, strains, in which the activities of ALD2 and ACS, or the activities of ALD3 and ACS were increased, were prepared based on the strain having a decreased PDC activity compared to that of a non-mutated microorganism. Specifically, strains having increased activities of ALD and ACS were prepared based on the strain having inactivated PDC1 via PDC1 defect and decreased PDC5 activity by substituting the gene promoter of PDC5 with a promoter having low expression ability. More specifically, strains of Saccharomyces sp. microorganism, in which PDC1 activity was inactivated, PDC5 activity was decreased, the activity at least one selected from the group consisting of ALD2 and ALD3 was increased, and ACS1 activity was increased, were prepared. Accordingly, it was confirmed that the growth rate of the strains, D-lactic acid production rate and the yield thereof were significantly improved.

The term “inactivation” of an enzyme activity of the present invention refers to a method for inactivating enzyme activities including any method that inhibits the expression of an enzyme, or allows the expression of an enzyme incapable of exhibiting its original activities. The method may include a partial gene deletion or a whole gene deletion caused by a homology recombination, an inhibition of an enzyme expression caused by an insertion of a foreign-derived gene into the relevant gene, an inhibition of an enzyme expression caused by a substitution or modification of a gene promoter sequence of the enzyme, or a mutation into an inactive-enzyme having a loss in its original functions caused by a substitution or modification of the enzyme, etc., but is not limited thereto.

The term “decrease” of an enzyme activity used herein refers to a method for decreasing the activity of an enzyme including any method for decreasing the expression level of an enzyme, or decreasing the activity of an enzyme being expressed. The method may include a decrease in an expression caused by a substitution or modification of a promoter sequence of the enzyme gene, or a mutation into an enzyme having decreased activity caused by a substitution or modification of the enzyme, etc., but is not limited thereto.

The term “increase” of an enzyme activity used herein refers to an insertion of a plasmid containing the genes of an enzyme, an increase in the number of gene copies encoding an enzyme on a chromosome, or an increase in an enzyme activity caused by a substitution or modification, or a mutation of a promoter sequence of an enzyme gene, etc., but is not limited thereto.

The term “yeast microorganism” used herein refers to a microorganism belonging to Eumycetes that proliferates by germination, but is not limited thereto as long as it is involved in any one of the lactic acid production pathway, alcohol production pathway, and/or acetyl-CoA production pathway. The yeast microorganism may be classified into Saccharomyces sp., Pichia sp., Candida sp., and Saccharomycopsis sp., depending on the shape of the yeast, and specifically, saccharomyces sp., which includes various species, may be applied in the present invention. Specifically, the microorganism may be selected from the group consisting of Saccharomyces bayanus, Saccharomyces boulardii, Saccharomyces bulderi, Saccharomyces cariocanus, Saccharomyces cariocus, Saccharomyces cerevisiae, Saccharomyces chevalieri, Saccharomyces dairenensis, Saccharomyces ellipsoideus, Saccharomyces eubayanus, Saccharomyces exiguus, Saccharomyces florentinus, Saccharomyces kluyveri, Saccharomyces martiniae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, and Saccharomyces zonatus, and more specifically, it may be Saccharomyces cerevisiae.

By preparing the microorganism having a decreased activity of PDC and improved activities of ALD and ACS based on Saccharomyces cerevisiae, a representative example of Saccharomyces sp., a significant increase in lactic acid production was confirmed.

The microorganism of the present invention may include alcohol dehydrogenase (ADH) that is to be further inactivated.

The term “alcohol dehydrogenase” used herein refers to a protein having an activity of catalyzing a reverse reaction responsible for producing aldehyde or ketone by removing hydrogen from alcohol, but is not limited to a derivative or an isotype having the same activity. Alcohol dehydrogenase of the present invention may be derived from Saccharomyces sp., or may be ADH1. Specifically, the protein may be ADH1 of Saccharomyces cerevisiae, but is not limited thereto, and may include any variant or an analogue thereof as long as they are biologically identical and have corresponding activities to the protein. The amino acid sequences of the protein may be obtained from a known database etc., e.g., GenBank of NCBI, etc., but is not limited thereto. Specifically, ADH1 may be composed of an amino acid sequence of SEQ ID NO: 77, and may include amino acid sequences having a homology of more than 70%, specifically more than 80%, more specifically more than 90%, and even more specifically more than 95%, to the amino acid sequence. A protein mutant of the sequence encoding the identical amino acid sequences, which results from genetic code degeneracy, may also be included in the present invention.

The microorganism of the present invention may include D-lactic acid dehyrogenase (DLD) that is further inactivated.

The term “D-lactic acid dehydrogenase” used herein, refers to a protein having an activity of producing pyruvate by anhydrization of D-lactic acid, but is not limited to an isotype having the same activity, D-lactic acid dehydrogenase of the present invention may be derived from Saccharomyces sp., Specifically, the protein may be DLD1 of Saccharomyces cerevisiae, but is not limited thereto, and may include any variant or an analogue thereof as long as they are biologically identical and have corresponding activities to the protein. The amino acid sequences of the protein may be obtained from a known database, etc., e.g., GenBank of NCBI, but is not limited thereto. Specifically, DLD1 may consist of an amino acid sequence of SEQ ID NO: 78, and may include amino acid sequences having a homology of more than 70%, specifically more than 80%, more specifically more than 90%, and even more specifically more than 95%, to the amino acid sequence. Any variant of the sequence encoding the identical amino acid sequences, which results from genetic code degeneracy, may also be included in the present invention.

In the present invention, the strains having a defect in ADH1, an enzyme involved in alcohol fermentation pathway using aldehyde as a substrate, which is further produced from pyruvate, and a defect in DLD1, an enzyme that decomposes the produced lactic acid, were used to precisely measure the changes in the lactic acid fermentation cell performances according to the regulation of acetic acid production pathway. In an exemplary embodiment of the present invention, the strains, in which the activities of PDC, ALD, and ACS were regulated, showed a significant increase in the lactic acid fermentation productivity. The results are summarized in Table 12.

In another aspect, the present invention provides a method for producing lactic acid using the microorganism of the present invention.

Specifically, in an exemplary embodiment of the present invention, the present invention provides a method for producing lactic acid including culturing the microorganism of the present invention and collecting lactic acid from the culture medium containing the microorganism.

The culturing may be performed using an appropriate medium and culturing conditions known in the art. According to the strains used, the culturing process may be readily adjusted by one of ordinary skill in the art. Examples of culturing methods include batch type, continuous type, and fed-batch type, but are not limited thereto. The media used in the culturing process should appropriately meet the requirements of a specific strain.

The medium used in the present invention contains sucrose or glucose as a main carbon source, and molasses containing a high concentration of sucrose may also be used as a carbon source. Other carbon sources may be used in an adequate amount variously. Organic nitrogen sources including peptone, yeast extract, meat extract, malt extract, corn steep liquor, and soybean wheat, and inorganic nitrogen sources including element, ammonium sulfate, ammonium chloride, ammonium phosphate, ammonium carbonate, and ammonium nitrate may be used as a nitrogen source. These nitrogen sources may be employed either singly or in combination. To the medium, phosphorus sources such as potassium dihydrogen phosphate, dipotassium hydrogen phosphate, or corresponding sodium-containing salts may be added. In addition, the medium may contain metal salts such as magnesium sulfate and iron sulfate. Further, the medium may be supplemented with amino acids, vitamins, and appropriate precursors. These media or precursors may be added to cultures by a batch type or continuous type method.

During the culturing process, compounds such as ammonium hydroxide, potassium hydroxide, phosphoric acid, and sulfuric acid may be properly added in order to adjust the pH of the culture. Further, a defoaming agent such as fatty acid polyglycol ester may be added in order to inhibit the formation of foams in the culture. In addition, to maintain the culture in an aerobic condition, oxygen or oxygen-containing gas may be injected into the culture, and to maintain the culture in anaerobic and micro-aerobic conditions, nitrogen, hydrogen, or carbon dioxide gases may be injected into the culture without injecting any gas.

The temperature of the culture may be maintained at 20 to 40° C., specifically at 25 to 35° C., and more specifically at 30° C. The culturing may be continued until a desired amount of the desired material is obtained, and specifically for 10 to 100 hours.

The lactic acid produced in the culturing processes of the present invention may be collected from the culture medium by a proper method known in the art, depending on the culturing method, e.g., batch type, continuous type, or fed-batch type.

### Advantageous Effects

The present invention relates to using a microorganism having improved lactic acid fermentation productivity by controlling the activities of PDC isotypes, and increasing the activities of aldehyde dehydrogenase (ALD) and acetyl-CoA synthetase (ACS). Therefore, it can be extensively used in the lactic acid fermentation production industries.

### BRIEF DESCRIPTION OF DRAWINGS

FIG. 1 is a schematic diagram illustrating the relationship between the lactic acid production pathway of a Saccharomyces sp microorganism, the alcohol fermentation pathway and the acetyl-CoA production pathway.

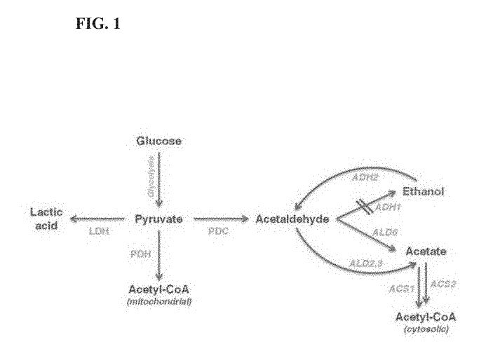

### BEST MODE FOR CARRYING OUT INVENTION

Hereinbelow, the present invention will be described in detail with accompanying exemplary embodiments. However, the exemplary embodiments disclosed herein are only for illustrative purposes and should not be construed as limiting the scope of the present invention.

### Example 1: Preparation of Lactic Acid-Producing Strain

To prepare lactic acid-producing strains, Saccharomyces cerevisiae CEN.PK2-1D, a representative wild type yeast obtained from EUROSCARF, was subject to genetic manipulation.

Specifically, a strain, where alcohol dehydrogenase 1 (ADH1) and pyruvate decarboxylase 1 (PDC1) were defective to minimize the loss of pyruvate to the alcohol synthesis pathway, and d-lactic acid dehydrogenase 1 (DLD1) was defective for blocking the D-type lactic acid decomposition pathway, was used as a base strain.

DLD1 is not a crucial factor that may have a direct impact on the growth improvement, but has been known as a major enzyme capable of converting D-lactic acid to pyruvate using NAD⁺ as D-lactic acid dehydrogenase. Accordingly, a subsequent strain was constructed based on the strain having gene defects in DLD1, an enzyme that consumes the prepared lactic acid thereof, to compare a complete fermentation productivity of D-type lactic acid-producing yeast, which is intended to be prepared in the present invention. As a result, the fermentation productivity was compared.

In the present invention, a general molecular cloning was employed for the gene manipulation.

First, in order to delete ADH1 and PDC1 genes of the yeast strains, an experiment was conducted with reference to the content disclosed in the Reference by Lee T H, et al. (J. Microbiol. Biotechnol. (2006), 16(6), 979-982), using plasmids pWAL100 and pWBR100. Each insert, which was introduced into the vector plasmids, was prepared using the suitable primers (corresponding to nucleotide sequences of SEQ ID NOS: 1 to 8) via PCR.

In addition, for the deletion of DLD1 gene, HIS3, which is a marker gene, was introduced by double crossover, and made it defective. The DNA fragments used therein were prepared using primers corresponding to nucleotide sequences of SEQ ID NOS: 9 and 10. The primers used in the gene manipulation are summarized in Table 1 below.

TABLE 1
Primers used for the
production of the base yeast strain

| Primer | 5′→3′ sequence |
|---|---|
| ADH1 upstream | CGGGATCCACTGTAGCCCTAGACTT |
| forward primer | GATAGCC |
| (SEQ ID NO: 1) | |
| ADH1 upstream | ATAAGAATGCGGCCGCTGTATATGA |
| reverse primer | GATAGTTGATTGTATGCTT |
| (SEQ ID NO: 2) | |
| ADH1 downstream | GACTAGTGCGAATTTCTTATGATTT |
| forward primer | ATGATTTTTATT |
| (SEQ ID NO: 3) | |
| ADH1 downstream | ACATGCCATGgAAGCATGCACGTAT |
| reverse primer | ACACTTGAGTAA |
| (SEQ ID NO: 4) | |
| PDC1 upstream | CGGGATCCATTATGTATGCTCTTCT |
| forward primer | GACTTTTCGT |
| (SEQ ID NO: 5) | |
| PDC1 upstream | ATAAGAATGCGGCCGCTTTGATTGA |
| reverse primer | TTTGACTGTGTTATTTTGC |
| (SEQ ID NO: 6) | |
| PDC1 downstream | CGGGATCCGCGATTTAATCTCTAAT |
| forward primer | TATTAGTTAAAG |
| (SEQ ID NO: 7) | |
| PDC1 downstream | ATAAGAATGCGGCCGCTTTCAATCA |
| reverse primer | TTGGAGCAATCATTTTACA |
| (SEQ ID NO: 8) | |
| DLD1-HIS3 | GCGTAGTTGGCCCCAACTGGTGCAG |
| upstream | TAATACGTTTTAAGAGCTTGGTGAG |
| linking primer | |
| (SEQ ID NO: 9) | |
| DLD1-HIS3 | CGTGAAGGGTGAAAAAGGAAAATCA |
| downstream | GATACCTACATAAGAACACCTTTGG |
| linking primer | |
| (SEQ ID NO: 10) | |

D-lactic acid dehydrogenase (D-LDH) specifically required for D-lactic acid production was introduced based on the strain having defects in the three genes such as ADH1, PDC1 and DLD1.

D-LDH was then cloned into a vector having restriction enzyme sites of XhoI and SpeI at 5′ and 3′ termini, respectively, in order for ldhD derived from lactobacillus plantarum (Lb. plantarum) to be included between TEF1 promoter derived from S. cerevisiae and CYC1 terminator. In particular, the insert was prepared by double-digestion of SacI/PvuII, and the vector was blunt ended by Mungbean nuclease from the DNA fragment, which was double-digested from p-δ-neo into BamHI/NotI. Lastly, the vector was treated with Sac I to thereby obtain a vector having a SacI sticky end and BamHI derived blunt end.

The construction of pTL573 vector was completed by the ligation of the obtained vector with the insert. The plasmid pTL573 contains the ldhD gene derived from Lb. plantarum, and it was designed so that it may include a random insertion of multiple copies of genes into partial domain of δ-sequence among retrotransposable element of S. cerevisiae CEN.PK2-1D pdc1Δ adh1Δ dld1Δ strain. For multiple insertion of a corresponding gene, DNA fragments capable of inducing single crossover on the δ-sequence were constructed by digesting plasmid pTL573 with SalI. By introducing the DNA fragments into a parent strain via transformation, a multiple colonies were obtained from YPD plate (1% yeast extract, 2% bacto-peptone, and 2% glucose) at a maximum concentration of 5 mg/mL G418. Finally, it was confirmed that the thus-obtained strain, the Lb. plantarum derived D-LDH, was multiply inserted for the purpose of providing D-lactic acid-producing-ability, and was assigned CC02-0064 strain.

### Example 2: Preparation of Mutant Strains Having Decreased PDC5 Activity

A mutant strain having substituted PDC5 promoter was prepared based on CC02-0064 strain prepared in Example 1. During the process, processes of cassette preparation and strain selection were conducted according to the method disclosed in Lee T. H. et al. (Development of reusable split URA3-marked knockout vectors for budding yeast, Saccharomyces cerevisiae. J Microbiol Biotechnol, 2006, 16:979-982).

Specifically, a total of five novel strains were prepared by substituting PDC5 promoter of the CC02-0064 strain with SCO1, SCO2, ACS1, IDP2, and FBA1 promoters, respectively, and subsequently, promoter-substituted cassettes were prepared using primers corresponding to nucleotide sequences of SEQ ID NOS: 11 to 36.

The primers used in the promoter substitution are summarized in Table 2 below.

TABLE 2
Primers used for the
preparation of promoter-substituted strains

| Primers | 5′→3′ sequence |
|---|---|
| F_PDC5_UP_676 | GTCAGCATTGACACGTTCGATT |
| (SEQ ID NO: 11) | |
| R_KlURA3-PDC5_UP | TCTACCCAGAATCACTTCTTTCGAGAGA |
| (SEQ ID NO: 12) | TTGTCATAATC |
| F_PDC5_UP- | CAATCTCTCGAAAGAAGTGATTCTGGGT |
| AL_KlURA3 | AGAAGATCGG |
| (SEQ ID NO: 13) | |
| R_AL_KlURA3 | GAGCAATGAACCCAATAACGAAATCTT |
| (SEQ ID NO: 14) | |
| F_BR_KlURA3 | CTTGACGTTCGTTCGACTGATGAG |
| (SEQ ID NO: 15) | |
| R_PDC5_DOWN_522 | CAAGTCAACCAAGTTAGCTGGC |
| (SEQ ID NO: 16) | |
| R_SCO1p-BR_KlURA3 | CTCTCCTAATAGACGTGGTGTCACCATG |
| (SEQ ID NO: 17) | AACGACAATTCTTAA |
| F_SCO1p_500 | CGTTCATGGTGACACCACGTCTATTAGG |
| (SEQ ID NO: 18) | AGAGCCATTC |
| R_PDC5_DOWN_500- | AAGGTTATTTCAGACATCTTTTCTACGT |
| SCO1p | TTGCTGTTTTTTC |
| (SEQ ID NO: 19) | |
| F_SCO1p- | CAGCAAACGTAGAAAAGATGTCTGAAAT |
| PDC5_DOWN_500 | AACCTTAGGTAAAT |
| (SEQ ID NO: 20) | |
| R_SCO2p-BR_KlURA3 | ATCGAATAAGTAACAAGCGTGTCACCAT |
| (SEQ ID NO: 21) | GAACGACAATTCTTAA |
| F_SCO2p_500 | CGTTCATGGTGACACGCTTGTTACTTAT |
| (SEQ ID NO: 22) | TCGATAACGC |
| R_PDC5_DOWN_500- | AAGGTTATTTCAGACATTTTACTCTCGC |
| SCO2p | TTCCCAAATTCC |
| (SEQ ID NO: 23) | |
| F_SCO2p- | GGAAGCGAGAGTAAAATGTCTGAAATAA |
| PDC5_DOWN_500 | CCTTAGGTAAAT |
| (SEQ ID NO: 24) | |
| R_IDP2p-BR_KlURA3 | TAAAAATAAATAGATAGACGTGTGTCAC |
| (SEQ ID NO: 25) | CATGAACGACAATTCTTAA |
| F_IDP2p_500 | CGTTCATGGTGACACACGTCTATCTATT |
| (SEQ ID NO: 26) | TATTTTTATAACTC |
| R_PDC5_DOWN_500- | AAGGTTATTTCAGACATTACGATTTTAT |
| IDP2p | ATATATACGTACGTTA |
| (SEQ ID NO: 27) | |
| F_IDP2p- | CGTATATATATAAAATCGTAATGTCTGA |
| PDC5_DOWN_500 | AATAACCTTAGGTAAAT |
| (SEQ ID NO: 28) | |
| R_ACS1p-BR_KlURA3 | CTGGACGTATGTGCACAGTGTCACCATG |
| (SEQ ID NO: 29) | AACGACAATTCTTAA |
| F_ACS1p_500 | CGTTCATGGTGACACTGTGCACATACGT |
| (SEQ ID NO: 30) | CCAGAATGAT |
| R_PDC5_DOWN_500- | AAGGTTATTTCAGACATAGCACAGTGGG |
| ACS1p | CAATGTCTTTC |
| (SEQ ID NO: 31) | |
| F_ACS1p- | CATTGCCCACTGTGCTATGTCTGAAATA |
| PDC5_DOWN_500 | ACCTTAGGTAAAT |
| (SEQ ID NO: 32) | |
| R_FBA1p-BR_KlURA3 | TTATTTACGTAATGACCCAGTGTCACCA |
| (SEQ ID NO: 33) | TGAACGACAATTCTTAA |
| F_FBA1p_500 | CGTTCATGGTGACACTGGGTCATTACGT |
| (SEQ ID NO: 34) | AAATAATGATAG |
| R_PDC5_DOWN_500- | AAGGTTATTTCAGACATTTTGAATATGT |
| FBA1p | ATTACTTGGTTATGGT |
| (SEQ ID NO: 35) | |
| F_FBA1p- | CCAAGTAATACATATTCAAAATGTCTGA |
| PDC5_DOWN_500 | AATAACCTTAGGTAAAT |
| (SEQ ID NO: 36) | |

The thus-prepared novel strains were assigned CC02-0167, CC02-0168, CC02-0169, CC02-0170, and CC02-0174, respectively. The corresponding strains and their genetic traits are summarized in Table 3 below.

TABLE 3
PDC5 promoter-mutated strains

| Strains | Genetic Traits |
|---|---|
| CC02-0167 | CC02-0064 PDC5 promoter::KlURA3-SCO1 promoter |
| CC02-0168 | CC02-0064 PDC5 promoter::KlURA3-SCO2 promoter |
| CC02-0169 | CC02-0064 PDC5 promoter::KlURA3-ACS1 promoter |
| CC02-0170 | CC02-0064 PDC5 promoter::KlURA3-IDP2 promoter |
| CC02-0174 | CC02-0064 PDC5 promoter::KlURA3-FBA1 promoter |

### Example 3: Evaluation of Lactic Acid Fermentation for Mutant Strains Having Decreased PDC5 Activity

An evaluation of lactic acid fermentation was conducted for the PDC5 promoter-mutated strains prepared in Example 2. In this regard, a specific medium was prepared for the evaluation of lactic acid fermentation.

Specifically, in order to prepare a synthetic complex media (SC media), a limiting medium for yeast, 0.67% yeast nitrogen base without amino acids serving as a base was mixed with amino acid dropout mix (Sigma) according to the protocol of the manufacturer, and added with the amino acids that were excluded in the base, as needed. In addition, 380 mg/L of leucine was added to the resultant, and uracil, tryptophan, and histidine were added at a concentration of 76 mg/L, respectively, 8% of glucose as a carbon source and 1% of CaCO₃ as a neutralizing agent were also added. The thus-prepared medium was used for the evaluation of lactic acid fermentation of the yeast strains.

Among the PDC5 promoter-mutated strains prepared in Example 2, the mutant strains substituted with a weaker promoter than the original PDC5 promoter failed to grow, whereas the mutant strains substituted with a stronger promoter showed improved growth. Specifically, the mutant strains substituted with promoters of SCO1, SCO2, IDP2 or ACS1, which are weaker promoters than PDC5 promoter, failed to grow, leaving the strains whose promoter was substituted with FBA1 promoter the only strains to be evaluated. The evaluation result of the lactic acid fermentation for CC02-0064 and CC02-0174 strains, which were measurable, is summarized in Table 4 below.

TABLE 4
Evaluation of lactic acid fermentation for PDC5 promoter-mutated strains

| | 24 hours | | | 48 hours | | | |
|---|---|---|---|---|---|---|---|
| | | Glucose | Lactic | | Glucose | Lactic | Yield |
| Strain | OD | Consumed | acid | OD | Consumed | acid | (%) |
| CC02- | 3.9 | 15.0 | 10.9 | 8.7 | 63.4 | 41.6 | 65.7 |
| 0064 | | | | | | | |
| CC02- | 5.7 | 25.0 | 19.8 | 9.4 | 69.9 | 47.3 | 67.7 |
| 0174 | | | | | | | |

As shown in the evaluation above, it was confirmed that, during the pathway promoting acetyl-CoA production, the strain where the wild-type PDC5 promoter was substituted with FBA1 promoter showed improved cell growth rate and lactic acid productivity thereof, compared to those of the original strain (CC02-0064). However, when the result of samples collected at 24 hours and 48 hours, respectively, were compared, it was confirmed that the improvements on the cell growth rate and the lactic acid productivity thereof according to the time were continued to reduce by a mere strengthening of a single PDC activity without strengthening ALD and ACS activities, which are involved in the subsequent acetyl-CoA producing pathway. In an example of the present invention, the improvement in the glucose consumption by the strengthening PDC activity was 10.3%, and the maximum lactic acid production concentration was 47.3 g/l. Accordingly, the overall improvement of the lactic acid productivity was 13.7%.

### Example 4: Preparation of a Strain Having a PDC5 Gene Defect

In addition to the strain having a PDC1 gene defect and decreased PDC5 activity prepared in Example 2, a strain having a defect in PDC5 gene and decreased PDC1 activity was prepared to thereby confirm whether PDC pathway was attenuated in the corresponding strain.

Specifically, for the purpose of a PDC5 gene defect, the primers corresponding to nucleotide sequences of SEQ ID NOS: 37 to 40 were used to prepare PDC5 gene defect cassette based on the CC02-0064 strain. The defective strain was prepared by the same method described the literature of Example 1. The primers used in Example 4 are summarized in Table 5 below.

TABLE 5
Primers used for the
preparation of the strain having PDC5 defects

| Primers | 5′→3′ sequence |
|---|---|
| F-ALPDC5- | GAGCTCGGATCCAAGGAAATAAAGCAAA |
| BamHI | TAACAATAACACC |
| (SEQ ID NO: 37) | |
| R-ALPDC5- | ACCATGGCGGCCGCTTTGTTCTTCTTGT |
| NotI | TATTGTATTGTGTTG |
| (SEQ ID NO: 38) | |
| F-BRPDC5- | GGATCCACTAGTGCTAATTAACATAAAA |
| SpeI | CTCATGATTCAACG |
| (SEQ ID NO: 39) | |
| R-BRPDC5- | CAGCTGCCATGGTATTCTAAATAAGATG |
| NcoI | TAAGGCCTTGTAAT |
| (SEQ ID NO: 40) | |

The thus-prepared strain having a PDC5 gene defect was assigned CC02-0450 (CC02-0064, pdc5Δ).

### Example 5: Preparation of PDC1 Promoter-Mutated Strains Based on the Strain Having a PDC5 Defect

A strain having substituted PDC1 promoter was prepared based on the CC02-0450 strain prepared in Example 4. In this regard, a strain CC02-0451 (CC02-0450, PDC1p-PDC1), where the defect in PDC1 gene was recovered, was prepared to serve as a comparative group, and a strain CC2-0452 (CC02-0450, IDP1p-PDC1) having decreased PDC1 activity was prepared to serve as an experimental group.

Each strain was prepared in such a way that the vectors of PDC1p-PDC1-CYC1t and pRS406-IDP2p-PDC1-CYC1, which were constructed by cloning a target gene cassette into a pRS406 vector without a replication origin in the yeast, to be included in the strain.

Specifically. PCR was conducted using primers having nucleotide sequences of SEQ ID NOS: 41 and 42 with chromosomal DNA of the yeast serving as a template, to thereby obtain a product including PDC1 gene. Subsequently, a sequence of CYC1 terminator was obtained using primers having nucleotide sequences of SEQ ID NOS: 43 and 44. In addition, DNA fragments connecting PDC1 and CYC1 terminator were obtained via PCR using primers corresponding nucleotide sequences of SEQ ID NOS: 41 and 44 with the PDC1 and the CYC1 terminator sequences, respectively, serving as a template. A plasmid vector of pRS406-PDC1-CYC1t was obtained by treating DNA fragments of PDC1-CYC1 terminator and pRS406 vector with SpeI and XhoI restriction enzymes followed by ligation thereof. Meanwhile, for the introduction of the promoter domain into the thus-obtained plasmid vectors, plasmid vectors, into which promoters of PDC1 and IDP2 promoters were respectively incorporated, were obtained by a primer fusion of primers having nucleotide sequences of SEQ ID NOS: 45 and 46, and 47 and 48, respectively, via PCR using chromosomal DNA as a template. DNA fragments including each promoter and pRS406-PDC1-CYC1t plasmid were digested and ligated, to thereby prepare plasmid vectors of pRS406-PDC1p-PDC1-CYC1t and pRS406-IDP2p-PDC1-CYC1t, respectively, which are plasmid required for the yeast chromosomal insertion, designed such that the gene expression is controlled by PDC1 promoter and IDP2 promoter.

The primers used in Example 5 are summarized in Table 6.

TABLE 6
Primers used for preparation of the strains
having a PDC5 defect and decreased PDC1 activity

| Primers | 5′→3′ sequence |
|---|---|
| F_PDC1 | ATAACTAGTATGTCTGAAATTACTTTG |
| (SEQ ID NO: 41) | GGTAAATATTT |
| R_PDC1 | CAAAGGAAAAGGGGCCTGTTTATTGCT |
| (SEQ ID NO: 42) | TAGCGTTGGTAGCAGCA |
| F_CYC1t | TACCAACGCTAAGCAATAAACAGGCCC |
| (SEQ ID NO: 43) | CTTTTCCTTTGTCGAT |
| R_CYC1t | ATACTCGAGGCAAATTAAAGCCTTCGA |
| (SEQ ID NO: 44) | GCGTCC |
| F_PDC1p | AAAGAGCTCCATGCGACTGGGTGAGCA |
| (SEQ ID NO: 45) | TATGTT |
| R_PDC1p | ATAACTAGTTTTGATTGATTTGACTGT |
| (SEQ ID NO: 46) | GTTATTTTGC |
| F_IDP2p | AAAGAGCTCACGTCTATCTATTTATTT |
| (SEQ ID NO: 47) | TTATAACTCC |
| R_IDP2p | ATAACTAGTTACGATTTTATATATATA |
| (SEQ ID NO: 48) | CGTACGTTAC |

The two thus-prepared plasmid vectors were digested by StuI, respectively, and inserted into the strains immediately. The final strains were assigned CC02-0451(CC02-0450, PDC1p-PDC1) and CC02-0452(CC02-0450, IDP2p-PDC1), respectively. The thus-prepared strains and their genetic traits are summarized in Table 7.

TABLE 7
Strains having PDC5 defect and decreased PDC1 activity

| Strains | Genetic Traits |
|---|---|
| CC02-0450 | CC02-0064 pdc5Δ |
| CC02-0451 | CC02-0450 PDC1p-PDC1-CYC1t |
| CC02-0452 | CC02-0450 IDP2p-PDC1-CYC1t |

### Example 6: Preparation of Strains Having Double or Triple Defects in PDC Genes

Strains having a single defect in PDC1 gene, a double defect in PDC1 and PDC5 genes, and a triple defect in PDC1, PDC5, and PDC6 genes were intended to be prepared from PDC family genes. CC02-0064 strain prepared in Example 1 was used as a strain having a single defect in PDC1 gene. A cassette for PDC5 defect was prepared using primers corresponding nucleotide sequences of SEQ ID NOS: 49 to 56, and inserted into CC02-0064 to prepare a strain having double defects in PDC1 and PDC5 genes. Subsequently, the thus-prepared strain was assigned CC02-0256. In addition, a strain having a triple defect in PDC1, PDC5, and PDC6 genes was prepared based on the strain having double defects in PDC1 and PDC5 genes using primers corresponding nucleotide sequence of SEQ ID NOS: 57 to 64 to, and was assigned CC02-0257.

The defect cassette preparation and strain selection process were conducted by the same method described in the literature disclosed in Example 1. The primers used in Example 6 are summarized in Table 8.

TABLE 8
Primers used for preparation of the strains
having double or triple defects in PDC genes

| Primers | 5′→3′ sequence |
|---|---|
| F_BamHI- | CGGGATCCAGGCCAAGGAAATAAAGCA |
| PDC5_UP | AATAACAA |
| (SEQ ID. 49) | |
| R_NotI-PDC5_UP | ATAAGAATGCGGCCGCTTTGTTCTTCT |
| (SEQ ID NO: 50) | TGTTATTGTATTGTGTT |
| F_BamHI- | CGGGATCCGCTAATTAACATAAAACTC |
| PDC5_DOWN | ATGATTCAA |
| (SEQ ID NO: 51) | |
| R_NotI- | ATAAGAATGCGGCCGCTATTCTAAATA |
| PDC5_DOWN | AGATGTAAGGCCTTGTA |
| (SEQ ID NO: 52) | |
| F_PDC5_UP | AGGCCAAGGAAATAAAGCAAATAACAA |
| (SEQ ID NO: 53) | |
| R_AL_KlURA3 | GAGCAATGAACCCAATAACGAAATCTT |
| (SEQ ID NO: 54) | |
| F_BR_KlURA3 | CTTGACGTTCGTTCGACTGATGAG |
| (SEQ ID NO: 55) | |
| R_PDC5_DOWN | TATTCTAAATAAGATGTAAGGCCTTGT |
| (SEQ ID NO: 56) | A |
| F_BamHI- | CGGGATCCTGTTATAGAGTTCACACCT |
| PDC6_UP | TATTCACA |
| (SEQ ID NO: 57) | |
| R_NotI-PDC6_UP | ATAAGAATGCGGCCGCTTTGTTGGCAA |
| (SEQ ID NO: 58) | TATGTTTTTGCTATATTA |
| F_BamHI- | CGGGATCCGCCATTAGTAGTGTACTCA |
| PDC6_DOWN | AACGAAT |
| (SEQ ID NO: 59) | |
| R_NotI- | ATAAGAATGCGGCCGCGATGCAGAATG |
| PDC6_DOWN | AGCACTTGTTATTTAT |
| (SEQ ID NO: 60) | |
| F_PDC6_UP | TGTTATAGAGTTCACACCTTATTCACA |
| (SEQ ID NO: 61) | |
| R_AL_KlURA3 | GAGCAATGAACCCAATAACGAAATCTT |
| (SEQ ID NO: 62) | |
| F_BR_KlURA3 | CTTGACGTTCGTTCGACTGATGAG |
| (SEQ ID NO: 63) | |
| R_PDC6_DOWN | GATGCAGAATGAGCACTTGTTATTTAT |
| (SEQ ID NO: 64) | |

The thus-prepared strains and their genetic traits are summarized in Table 9.

TABLE 9
Strains having double or triple defects in PDC genes

| Strains | Genetic traits |
|---|---|
| CC02-0256 | CC02-0064 pdc5Δ |
| CC02-0257 | CC02-0256 pdc6Δ |

### Example 7: Preparation of ALD and ACS1 Overexpressing Strains

For the preparation of ALD and ACS1 over expressing strains, ALD2, ALD3, and ACS1 over expressing plasmids were prepared.

Specifically, an open reading frame (ORF) of ALD2 was prepared using primers corresponding nucleotide sequences of SEQ ID NOS: 65 and 66, an ORF of ALD3 was prepared using primers corresponding nucleotide sequences of SEQ ID NOS: 67 and 68, and an ORF of ACS1 was prepared using primers corresponding nucleotide sequences of SEQ ID NOS: 69 and 70. In addition, p415ADH-ALD2, p415ADH-ALD3, p414ADH-ACS1 and p416ADH-ACS1, which are p414ADH, p415ADH and p416ADH plasmid-based recombinant vectors, were prepared by SpeI, XhoI or EcoRI restriction enzymes. The primers used in Example 7 are summarized in Table 10 below.

TABLE 10
Primers used for the preparation
of ALD and ACS1 overexpressing strains

| Primers | 5′→3′ sequence |
|---|---|
| F_SpeI_ALD2 | CAAGCTGGCCGCTCTAGAACTAGTATGC |
| (SEQ ID NO: 65) | CTACCTTGTATACTGATATCGA |
| R_XhoI_ALD2 | ACATAACTAATTACATGACTCGAGTTAG |
| (SEQ ID NO: 66) | TTGTCCAAAGAGAGATTTATGT |
| F_SpeI_ALD3 | CAAGCTGGCCGCTCTAGAACTAGTATGC |
| (SEQ ID NO: 67) | CTACCTTGTATACTGATATCGA |
| R_XhoI_ALD3 | ACATAACTAATTACATGACTCGAGTTAT |
| (SEQ ID NO: 68) | TTATCCAATGAAAGATCCACAT |
| F_SpeI_ACS1 | TCCAAGCTGGCCGCTCTAGAACTAGTAT |
| (SEQ ID NO: 69) | GTCGCCCTCTGCCGTACA |
| R_EcoRI_ACS1 | TATCGATAAGCTTGATATCGAATTCTTA |
| (SEQ ID NO: 70) | CAACTTGACCGAATCAATTAGA |

The thus-prepared recombinant plasmids were introduced into the strains including CC02-0064, CC02-0168, CC02-0170, CC02-0256, CC2-0257, CC02-0451, and CC02-0452 via a yeast transformation by p415ADH-ALD2, p414ADH-ACS1 combination, p415ADH-ALD3, p414ADH-ACS1 combination, p415ADH-ALD2, p416ADH-ACS1 combination, or p415ADH-ALD3, p416ADH-ACS1 combination. However, no transformant was obtained in the CC02-0257 strain having triple defects in PDC genes where no PDC activity was exhibited.

The thus-prepared strains and their genetic traits and summarized in Table 11.

TABLE 11
ALD and ACS overexpressing strains

| Strains | Genetic Traits |
|---|---|
| CC02-0225 | CC02-0064 p415ADH, p416ADH |
| CC02-0226 | CC02-0064 p415ADH-ALD2, p416ADH-ACS1 |
| CC02-0227 | CC02-0064 p415ADH-ALD3, p416ADH-ACS1 |
| CC02-0356 | CC02-0168 p414ADH, p415ADH |
| CC02-0275 | CC02-0168 p414ADH-ACS1, p415ADH-ALD2 |
| CC02-0276 | CC02-0168 p414ADH-ACS1, p415ADH-ALD3 |
| CC02-0357 | CC02-0170 p414ADH, p415ADH |
| CC02-0277 | CC02-0170 p414ADH-ACS1, p415ADH-ALD2 |
| CC02-0278 | CC02-0170 p414ADH-ACS1, p415ADH-ALD3 |
| CC02-0444 | CC02-0256 p415ADH, p416ADH |
| CC02-0361 | CC02-0256 p415ADH-ALD2, p416ADH-ACS1 |
| CC02-0362 | CC02-0256 p415ADH-ALD3, p416ADH-ACS1 |
| CC02-0453 | CC02-0451 p414ADH, p415ADH |
| CC02-0454 | CC02-0451 p414ADH-ACS1, p415ADH-ALD2 |
| CC02-0455 | CC02-0451 p414ADH-ACS1, p415ADH-ALD3 |
| CC02-0456 | CC02-0452 p414ADH, p415ADH |
| CC02-0457 | CC02-0452 p414ADH-ACS1, p415ADH-ALD2 |
| CC02-0458 | CC02-0452 p414ADH-ACS1, p415ADH-ALD3 |

### Example 8: Evaluation of Lactic Acid Fermentation for the Yeast Strains

An evaluation of lactic acid fermentation-ability for the ALD and ACS1 overexpressing strains, prepared in Example 7, was conducted.

Specifically, the yeast was inoculated into each flask containing 25 ml of the medium, prepared in Example 3 for the purpose of lactic acid fermentation evaluation and was cultured under aerobic condition at 30° C. for 71 hours. The amount of D-type lactic acid present in the fermented broth was analyzed, and an enzymatic analysis (Acetic acid, R-Biopharm, Germany) was conducted to determine the amount of acetic acid present therein.

The above experiment results are summarized in Table 12 below.

TABLE 12
Evaluation of the growth rate, lactic acid fermentation, by-products, and
production yield, etc., for the ALD and ACS overexpressing strains

| | | Initial | Residual | | D-lactic | | Produc- |
|---|---|---|---|---|---|---|---|
| | Final | glucose | glucose | Acetate | acid | Yield | tivity |
| Strains | OD | (g/L) | (g/L) | (g/L) | (g/L) | (g/g) | (g/l · h) |
| CC02- | 9.3 | 88 | 10 | 2.87 | 41.1 | 0.53 | 0.579 |
| 0225 | | | | | | | |
| CC02- | 9.3 | 83 | 10.5 | 2.91 | 42.4 | 0.59 | 0.597 |
| 0226 | | | | | | | |
| CC02- | 10.1 | 84 | 9.5 | 2.91 | 41.8 | 0.56 | 0.589 |
| 0227 | | | | | | | |
| CC02- | 6.9 | 88 | 26 | 0.02 | 27.6 | 0.45 | 0.389 |
| 0356 | | | | | | | |
| CC02- | 11.6 | 88 | 11.5 | 0.01 | 47.6 | 0.62 | 0.670 |
| 0275 | | | | | | | |
| CC02- | 10.6 | 88 | 11 | 0.01 | 46.8 | 0.61 | 0.659 |
| 0276 | | | | | | | |
| CC02- | 12.2 | 88 | 13 | 0.04 | 38.1 | 0.51 | 0.537 |
| 0357 | | | | | | | |
| CC02- | 17.8 | 88 | 1 | 0.03 | 58.6 | 0.67 | 0.825 |
| 0277 | | | | | | | |
| CC02- | 18.8 | 88 | 0 | 0.02 | 56.9 | 0.66 | 0.801 |
| 0278 | | | | | | | |
| CC02- | 9.8 | 88 | 8.5 | 2.2 | 38.9 | 0.49 | 0.548 |
| 0453 | | | | | | | |
| CC02- | 10.2 | 88 | 8.1 | 2.4 | 39.5 | 0.49 | 0.556 |
| 0454 | | | | | | | |
| CC02- | 9.2 | 88 | 8.8 | 2.1 | 40 | 0.51 | 0.563 |
| 0455 | | | | | | | |
| CC02- | 12 | 88 | 10.1 | 0.02 | 38.5 | 0.49 | 0.542 |
| 0456 | | | | | | | |
| CC02- | 18.1 | 88 | 0 | 0.02 | 55.8 | 0.63 | 0.786 |
| 0457 | | | | | | | |
| CC02- | 18.5 | 88 | 0 | 0.02 | 56.5 | 0.64 | 0.800 |
| 0458 | | | | | | | |

As verified in Table 12, the strains having decreased PDC5 activity by IDP2 promoter or SCO2 promoter had a dramatic reduction in the accumulation of the acetate, a by-product, i.e., little detection of acetate was confirmed, compared to the strain with normal PDC5 activity. In such case, the final cell concentration of the strains, in which the activities of ALD and ACS were not increased, tended to decrease according to the PDC5 promoter substitutions. On the contrary, the strains, where the PDC5 expression was reduced and the activities of ALD and ACS were increased, showed an increase in the final cell concentration. Accordingly, the improvement in the cell growth was confirmed. Specifically, the strains of CC02-0277 and CC02-0278 having increased ALD and ACS activities prepared based on the CC02-0170 strain, where PDC5 promoter was substituted with IDP2, showed improved growth rate, D-lactic acid concentration of production, production yield thereof, and fermentation productivity as increasing in the ALD and ACS activities.

In summary, the strain where PDC5 was substituted with a weak expression of IDP2 had a reduction in acetate accumulation, and the final OD was 1.3 times higher compared to the strain exhibiting normal expression of PDC5. In addition, it was confirmed that, when ALD and ACS were co-expressed under the control of the ADH1 promoter, the glucose consumption and the rate thereof were increased, and finally, the percentage yield was increased from 56% or %59% to 66% or 67%, respectively, showing improved yield.

Specifically, by comparing the two kinds of promoters applied to the weak expression of PDC5, it was confirmed that, the lactic acid productivity was improved in both strains having SCO2 promoter and IDP2 promoter, respectively. However, the strain having IDP2 promoter may be considered as the most optimized form of a strain in terms of the overall cell concentration, the glucose consumption, and the rate thereof.

### Example 9: Evaluation of Lactic Acid Fermentation for the Yeast Strain Having Double Defects in PDC1 and PDC5 Genes, and Increase in ALD and ACS Activities

Since the effects of the cell growth and the yield improvements resulted from the decreased PDC5 activity have been clearly confirmed, an evaluation was undertaken to determine the effects of additional PDC gene defect in lactic acid production. The evaluation method for each strain was identical to the method described in Example 8, and the culturing was conducted for 74 hours.

The thus-obtained experiment results are summarized in Table 13 below.

TABLE 13
The evaluation results of lactic acid fermentation for the strains having
double defects in PDC1 and PDC5 genes

| | | Initial | Residual | | D-lactic | | Produc- |
|---|---|---|---|---|---|---|---|
| | Final | Glucose | Glucose | Acetate | acid | Yield | tivity |
| Strains | OD | (g/L) | (g/L) | (g/L) | (g/L) | (g/g) | (g/l · h) |
| CC02- | 3.2 | 78.5 | 52 | 0.10 | 20.8 | 78.5 | 0.281 |
| 0444 | | | | | | | |
| CC02- | 3.9 | 78.5 | 49 | 0.05 | 25.4 | 86.3 | 0.343 |
| 0361 | | | | | | | |
| CC02- | 3.8 | 78.5 | 51 | 0.08 | 22.8 | 82.8 | 0.308 |
| 0362 | | | | | | | |

As confirmed in Table 13, the acetate concentration was clearly reduced in the strains having double defects in PDC1 and PDC5 genes, however, a reduction in the D-lactic acid concentration of production was also observed due to the reduction in the cell growth and the glucose consumption. In addition, the strain where the PDC pathway is almost inactivated, which was resulted from the double defects in PDC1 and PDC5 genes, did not have any improvement in the cell growth, the glucose consumption, and the productivity thereof, although the strain exhibited increased activities of ALD and ACS.

### Example 10: Evaluation of Lactic Acid Fermentation for the Strains, where PDC Pathway is Attenuated, Using Sucrose

For the purpose of a fermentation evaluation using sucrose, the lactic acid-producing yeast strains, where PDC pathway is attenuated, the identical strains evaluated in Example 8 and 9 were used to confirm the effect of the lactic acid production. In this regard, sucrose was employed as a carbon source instead of glucose. The evaluation method was performed in the same manner as Example 8.

The thus-obtained experiment results are summarized in Table 14 below.

TABLE 14
The evaluation results of lactic acid fermentation for the strains having
double defects in PDC1 and PDC5 genes or decreased PDC5 activity

| | | Initial | Residual | | D-lactic | | Produc- |
|---|---|---|---|---|---|---|---|
| | Final | Glucose | Glucose | Acetate | acid | Yield | tivity |
| Strains | OD | (g/L) | (g/L) | (g/L) | (g/L) | (g/g) | (g/l · h) |
| CC02- | 5.15 | 91.5 | 27.5 | 1.95 | 25.19 | 39.36 | 0.34 |
| 0225 | | | | | | | |
| CC02- | 6.9 | 91.5 | 15 | 1.92 | 30.89 | 40.38 | 0.42 |
| 0226 | | | | | | | |
| CC02- | 6.18 | 91.5 | 15 | 1.98 | 29.59 | 38.68 | 0.40 |
| 0227 | | | | | | | |
| CC02- | 1.6 | 91.5 | 26.75 | 0.02 | 11.72 | 18.11 | 0.16 |
| 0356 | | | | | | | |
| CC02- | 1.88 | 91.5 | 21.75 | 0.02 | 13.79 | 19.77 | 0.19 |
| 0275 | | | | | | | |
| CC02- | 2.2 | 91.5 | 18.25 | 0.01 | 16.04 | 21.9 | 0.22 |
| 0276 | | | | | | | |
| CC02- | 2.78 | 91.5 | 22 | 0.03 | 17.08 | 24.58 | 0.23 |
| 0357 | | | | | | | |
| CC02- | 12.15 | 91.5 | 7.75 | 0.02 | 44.65 | 53.31 | 0.60 |
| 0277 | | | | | | | |
| CC02- | 11.88 | 91.5 | 6.25 | 0.01 | 43.84 | 51.43 | 0.60 |
| 0278 | | | | | | | |
| CC02- | 2.45 | 91.5 | 37 | 0.02 | 21.94 | 40.25 | 0.30 |
| 0444 | | | | | | | |
| CC02- | 2.5 | 91.5 | 18.25 | 0.02 | 21.73 | 29.66 | 0.29 |
| 0361 | | | | | | | |
| CC02- | 3.08 | 91.5 | 15.25 | 0.02 | 24.92 | 32.67 | 0.34 |
| 0362 | | | | | | | |

The use of sucrose instead of glucose for the strains, used in the same manner as in Examples 8 and 9, allowed for improved effects of the growth and fermentation yield by increasing the activities of ALD and ACS in the strains where PDC pathway is attenuated, showing the same pattern of results as the strains in Example 8 where glucose was employed as a carbon source. Accordingly, the present invention confirms that the improved effects of fermentation yield and growth due to the decreased PDC activity and increased ALD and ACS activities, which were confirmed in the present invention, are not limited to the type of sugar used.

To summarize the above results, it was confirmed that, when the strains were mutated in such a way that PDC pathway was attenuated, and the activities of ALD and ACS were improved compared to that of the non-mutated strains, the lactic acid production was increased, and the growth rate thereof was maintained simultaneously.

From the foregoing, a skilled person in the art to which the present invention pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. On the contrary, the present invention is intended to cover not only the exemplary embodiments but also various alternatives, modifications, equivalents and other embodiments that may be included within the spirit and scope of the present invention as defined by the appended claims.

1. An isolated Saccharomyces cerevisiae microorganism having enhanced productivity of lactic acid, wherein the microorganism is modified so that:
a) the activity of pyruvate decarboxylase (PDC) of the microorganism is decreased compared to that of a non-modified lactic acid-producing strain; and
b) the activities of aldehyde dehydrogenase (ALD) and acetyl-CoA synthetase (ACS) of the microorganism are enhanced compared to that of the non-modified lactic acid-producing strain.

2. The microorganism according to claim 1, wherein the pyruvate decarboxylase is at least one selected from the group consisting of PDC1, PDC5, and PDC6.

3. The microorganism according to claim 2, wherein the microorganism is modified to:
i) inactivate PDC1 activity and decrease PDC5 activity; or
ii) decrease PDC1 activity and inactivate PDC5 activity.

4. The microorganism according to claim 1, wherein the aldehyde dehydrogenase is at least one selected from the group consisting of ALD2 and ALD3, and the acetyl-CoA synthetase is ACS1.

5. The microorganism according to claim 1, wherein alcohol dehydrogenase (ADH) is further inactivated.

6. The microorganism according to claim 1, wherein D-lactic acid dehydrogenase (DLD) is further inactivated.

7. A method for producing lactic acid comprising:
a) culturing the microorganism according to claim 1 in the culture medium; and
b) recovering lactic acid from the culture medium or the microorganism in step a).

8. A method for producing lactic acid comprising:
a) culturing the microorganism according to claim 2 in the culture medium; and
b) recovering lactic acid from the culture medium or the microorganism in step a).

9. A method for producing lactic acid comprising:
a) culturing the microorganism according to claim 3 in the culture medium; and
b) recovering lactic acid from the culture medium or the microorganism in step a).

10. A method for producing lactic acid comprising:
a) culturing the microorganism according to claim 4 in the culture medium; and
b) recovering lactic acid from the culture medium or the microorganism in step a).

11. A method for producing lactic acid comprising:
a) culturing the microorganism according to claim 5 in the culture medium; and
b) recovering lactic acid from the culture medium or the microorganism in step a).

12. A method for producing lactic acid comprising:
a) culturing the microorganism according to claim 6 in the culture medium; and
b) recovering lactic acid from the culture medium or the microorganism in step a).
